(19) United States
(12) Patent Application Publication
(10) Pub. No.: US 2013/0273011 A1
(43) Pub. Date: Oct. 17, 2013

(54) STEM CELLS AND STEM CELL GENERATED NANOPARTICLES FOR TREATMENT OF INFLAMMATORY CONDITIONS AND ACUTE RADIATION SYNDROME

(71) Applicant: MEDISTEM, INC., San Diego, CA (US)

(72) Inventors: Thomas Ichim, San Diego, CA (US); Vladimir Bogin, Portland, OR (US); Amit Patel, Salt Lake City, UT (US)

(73) Assignee: Medistem, Inc., San Diego, CA (US)

(21) Appl. No.: 13/865,103

(22) Filed: Apr. 17, 2013

Related U.S. Application Data
(60) Provisional application No. 61/625,657, filed on Apr. 17, 2012.

(51) Int. Cl. A61K 35/28

(52) U.S. Cl. CPC A61K 35/28
USPC 424/93.7

(57) ABSTRACT

Disclosed are stem cells and nanoparticles derived from said stem cells, useful for the treatment of acute radiation syndrome (ARS) and other inflammatory conditions. In one embodiment, Endometrial Regenerative Cells (ERC) are administered to patients having been exposed to radiation to augment recovery of endogenous hematopoietic stem cells. In another embodiment, exosomes, nanoparticles that are generated by various stem cells are used as a drug for treatment of inflammatory conditions.

### CROSS-REFERENCE TO RELATED APPLICATIONS

This application claims priority to Provisional Application Ser. No. 61/625,657, filed Apr. 17, 2012, and entitled “Stem Cells and Stem Cell Generated Nanoparticles for Treatment of Inflammatory Conditions and Acute Radiation Syndrome” which is hereby expressly incorporated by reference in its entirety.

### FIELD OF THE INVENTION

The invention pertains to the field of stem cell therapeutic activities, more particularly the invention pertains to treatment of hematopoietic injuries through administration of mesenchymal stem cells that are capable of secreting trophic factors capable of increasing survival after radiation injury through accelerating hematopoietic recovery, more particularly the invention also pertains to the use of exosomes secreted by said mesenchymal stem cells for achievement of this purpose.

### BACKGROUND

Protection against accidental or terrorist radiation exposure is attracting an increasing attention from military and civilian groups [1]. The possibility of nuclear war remains a reality: currently there are approximately 30,000 nuclear warheads deployed around the world, 100 “suitcase bombs” unaccounted for, and attempts of terrorists to acquire a nuclear weapon, a “dirty bomb” or to attack a nuclear power plant or waste site. A Nuclear Regulatory Commission study stated that breaching a cask of spent fuel could release lethal radiation over an area many times larger than that affected by a 10 kiloton nuclear weapon [2]. Acute Radiation Syndrome (ARS), which is the main cause of morbidity and mortality associated with ionizing radiation exposure is characterized by the triad of dysfunctions in the: a) neurovascular; b) hematopoietic; and c) gastrointestinal systems [3]. Intermediate dose ARS, which is similar to that received by firefighters at the Fukushima Daiichi Nuclear Power Plant and (3 to 7 Gy total body irradiation) is generally treated with hematopoietic growth factor support, whereas, high dose 7-10 (Gy) is treated experimentally with hematopoietic stem cell transplant [4]. Of the three systems that ARS targets, by far the most work has been performed in hematopoietic recovery with specific guidelines in place for administration of growth factors such as G-CSF and GM-CSF post exposure [3]. To date, with exception of potassium iodine, there is only one drug that has been FDA approved for post-radiation exposure, amifostine, which acts as a DNA protectant and antioxidant [5]. Unfortunately, its administration is associated with a variety of adverse effects including hypotension in >60% of patients, and its radioprotectant effects are limited in cases of myeloablative radiation [6]. Accordingly, there is a need in the art to develop novel means of addressing radiation exposure, particularly in the case of acute radiation syndrome.

### SUMMARY OF THE INVENTION

In 2007 we discovered a novel subset of mesenchymal stem cells (MSC) derived from the endometrium, termed “Endometrial Regenerative Cells (ERC). In comparison to other MSC types (eg bone marrow and adipose), ERC possess: a) more rapid proliferative rate; b) higher levels of growth factor production (VEGF, GM-CSF, PDGF) and c) higher angiogenic activity. We are currently running two clinical trials for these cells in patients with critical limb ischemia and heart failure. The main cause of morbidity and mortality in patients suffering from Acute Radiation Syndrome (ARS) is hematopoietic toxicity, although pulmonary fibrosis, neurovascular complications, and gastrointestinal injury are also significant contributing factors to morbidity and mortality. Recent studies have demonstrated that BM-MSC are capable of preventing lethality subsequent to radiation exposure, however, these cells have performed poorly in late-phase trials. Given that ERC are substantially more economical to manufacture in large numbers, and produce more hematopoietically-relevant factors as compared to other MSC sources, in one aspect, the invention provides the use of endometrial regenerative cells (ERC) administered intravenously to treat patients subsequent to radiation exposure.

Exosomes are nanoparticles generated by a variety of cell types, implicated in cell to cell communication. MSC-BM exosomes have been shown to be a major mediator of MSC paracrine therapeutic effects. Our data demonstrate that ERC-generated exosomes stimulate BM mononuclear cell proliferation. Accordingly, in one aspect of the invention, ERC derived exosomes are used as a therapeutic for radiation exposure.

### BRIEF DESCRIPTION OF THE DRAWINGS

FIG. 1 is a graph showing the stimulation of bone marrow mononuclear cell proliferation by ERC and BM-MSC Exosomes.

### DETAILED DESCRIPTION OF THE ILLUSTRATED EMBODIMENTS

In one embodiment, the invention teaches that ERC, a type of MSC, are useful for treatment of ARS. ERC are a type of MSC that possess superior growth factor production activity, as well as enhanced ability to stimulate proliferation of bone marrow mononuclear cells. In one embodiment, the invention provides a “second generation” MSC that is useful and practical for the treatment of ARS.

MSC are known to contribute to the bone marrow hematopoietic microenvironment. Given that this microenvironment is disrupted by radiation damage [7, 8], studies were conducted to demonstrate that human MSC can accelerate hematopoietic reconstitution and/or recovery in animal models [9, 10]. Therapeutic activities of MSC are believed to occur by differentiating into cells of mesenchymal origin [11], and also through an indirect “chaperone” effect. This includes production of trophic/angiogenic factors, as well as anti-inflammatory/anti-oxidant properties [12, 13]. One interesting aspect of MSC is that production of growth factors such as IGF-1, VEGF, and HGF-1 seems to be upregulated by conditions associated with injury such as hypoxia [14], and inflammatory conditions [15, 16]. Supporting possible use of MSC in treatment of radiation injuries are findings that MSC specifically home to areas of radiation exposure [17]. In preclinical studies it has been demonstrated that human MSC administration enhances engraftment of human CD34 cells post radiation [10]. Accordingly, clinical implications of using MSC administration to enhance hematopoiesis were examined. Original clinical use of expanded autologous MSC in 1995 demonstrated feasibility and safety of intravenous administration of these cells in 15 patients suffering from various hematological malignancies to prevent cytopenia [18]. In a subsequent study from the same group in 2000, the use of MSC to accelerate hematopoietic reconstitution was performed in a group of 28 breast cancer patients who received high dose chemotherapy [19]. Donor MSC were demonstrated to neutrophil and thromocytic reconstitution in a post bone marrow transplant setting in a 46 patient trial [20]. In a similar study, Ball et al reported on use of purified donor-specific MSC (1-5 million/kg) being injected alongside with isolated CD34 from HLA-mismatched relatives in 14 pediatric leukemia patients. They showed that in contrast to traditional graft failure rates of 15% in 47 historical controls, all patients given MSCs showed sustained hematopoietic engraftment without any adverse reaction [21]. Use of “third-party” MSC to enhance peripheral blood stem cell grafts was performed by Baron et al in 20 patients who received non-myeloablative hematopoietic stem cell transplant, whose outcomes were compared to a historic control of 16 patients receiving a similar transplant protocol without MSC accelerated hematopoietic reconstitution and significant difference in 1 year survival (80% vs 44%) was noted [22]. These established a foundation for MSC-based therapies to be investigated clinically as augmenters of hematopoietic reconstitution and/or prevention of GVHD, with Phase II/III trials ongoing or having been completed [23]. Unfortunately, bone marrow derived MSC have previously failed in various Phase II and Phase III trials. The invention provides the use of ERC to overcome shortcomings of BM-MSC in the treatment of ARS.

In addition to preclinical and clinical data supporting use of MSC in acceleration of hematopoietic reconstitution, several animal studies have formally studied ARS protection by MSC. These studies have yielded promising results, however clinical translation of ERC for use in ARS has not occurred. Yang et al demonstrated that a onetime infusion of either virally immortalized or primary mouse BM-MSC (1 million cells per mouse i.v.) 24 hours subsequent to 700 cGy X-radiation exposure led to a 53% survival in mice receiving immortalized and 60% survival for the group receiving primary MSC at 7 weeks post irradiation. Mice that were treated with vehicle control all died [24]. Lange et al obtained similar results in that administration of 1 million cloned or primary BM-MSC into lethally irradiated (9.5 Gy from Cesium-137 source) 8 hours after radiation resulted in 7-week survival of 66% of treated animals, whereas 100% of control animals died within 3 weeks. Although administered cells were localized primarily in the lung, microarray detection of gene expression in the bone marrow was noted, particularly, upregulation of genes associated with cell cycle and protection from oxidative stress, such as Cdkn1a and BRPK, as well as anti-inflammatory and detoxification genesThbs2 and Gstm5. Survival was associated with reconstitution of endogenous hematopoiesis [25]. Similar results were reported by another group, in which it was demonstrated that BM-MSC infusion after sublethal irradiation (5.5 Gy) was associated with enhanced survival of BALB/c mice, as well as stimulation of bone marrow cell entry into cell cycle and reduction of apoptosis [26]. In one embodiment, one of skill in the art will use the mentioned examples of assessing a potential treatment for ARS in the optimization of ERC for use with the practice of the invention.

Although bone marrow failure is the major cause of morbidity and mortality, in “real life” situations, ARS would likely be accompanied by GI failure, neurological consequences, pulmonary fibrosis and possibility of multiorgan failure. This was observed in victims of the Chernobyl Nuclear Accident [27, 28]. Protection of the GI tract and 100% 3 week survival subsequent to 10.4 Gy whole body radiation exposure was demonstrated in mice treated with BM cells cultured in an MSC-differentiation media, whereas 100% mortality occurred in controls [29]. MSC have been demonstrated to be neuroprotective in models of stroke [30], intracerebral hemorrhage [31], as well as having ability to stimulate endogenous neurogenesis [32]. Although to date studies on MSC prevention of radiation-induced neural damage have not been performed, given that radiation inhibits endogenous neurogenesis [33], this is an appealing possibility. MSC have been demonstrated to inhibit pulmonary fibrosis through anti-inflammatory mechanisms in several models [34]. Furthermore, multiorgan failure, whether induced by radiation or sepsis, presents with similar qualities. Inhibition of sepsis associated multiorgan failure has been demonstrated by BM-MSC and appears to function through an IL-10 and PGE-2 dependent pathway [35].

ERC as defined within the context of the invention are menstrual-blood derived MSC population that possesses a higher proliferative rate (19-22 hours), increased growth/angiogenic factor production, and longer passage ability as compared to BM-MSC [36]. These properties, as well as enhanced antifibrotic activities, were confirmed by 2 independent groups a year after [37, 38]. ERC are the subject of our patent application “Endometrial stem cells and methods of making and using same”. # 20090053182, and incorporated by reference. Currently we are conducting a Phase I study in critical limb ischemia (NCT01558908) in the USA, and a Phase II double blind-placebo controlled cardiac study Ex-USA.

ERC populations within the context of the invention, have to date been administered via intrathecal [39], intramuscular [40], and intravenous routes [41], in pilot compassionate-use cases. This has revealed feasibility of administration. The use of ERC for ARS is relevant not only because of potential benefit based on enhanced antifibrotic and growth factor production properties of ERC compared to other MSC populations, but also due to low cost of isolation and mass production ($500 per clinical dose of 100 million cells).

In one embodiment of the invention, exosomes are utilized as a therapeutic drug for treatment of ARS. Exosomes are nanoparticles (40-100 nm) in size that possess highly defined homogeneous characteristics [42]. In one embodiment, exosomes from mesenchymal stem cell populations are utilized for treatment of ARS. Specifically, exosomes derived from MSC may be administered intravenously, intrathecally, intranasally, by aerosol, by suppository, or subcutaneously. In one embodiment, exosomes generated by ERC are used as a superior source in comparison to those produced by BM MSC for treatment of ARS. In other embodiments, exosomes generated from MSC are used in the treatment of chronic obstructive pulmonary disease. In a specific embodiment exosomes generated from ERC are administered intravenously or by aerosol delivery as a treatment for COPD.

Methods of isolating exosomes, as well as for analyzing exosomes for activity, are known in the art and incorporated by reference. For example, in the art there are numerous citations that provide such techniques from other cells such as T cells [43, 44], B cells [45, 46], dendritic cells [47, 48], tumor cells [49, 50], neurons [51, 52], oligodendrocytes [53], and placental cells [54]. Techniques specific to exosomes derived from MSC or CD34 cells have also been disclosed in studies which have demonstrated that stem cell derived exosomes are responsible at least in part for paracrine angiogenic and cardioprotective activity of cell therapy products such as MSC [55, 56]. In one embodiment of the invention, techniques referenced are utilized to guide one of skill in the art to generate therapeutic compositions for treatment of ARS. Said exosomal compositions are also useful for the treatment of of other inflammatory conditions, within the current invention the use of ERC exosomes for treatment of chronic obstructive pulmonary disease (COPD) is provided. In this manner, ERC or other MSC exosomes are administered by aerosol inhalation or intranasally. This administration method delivers large amounts of ERC directly to the target lung tissue. In other embodiments ERC exosomes are given by inhalation for pulmonary fibrosis associated with radation., Given that exosomes can be produced en masse in a bioreactor setting, and that safety and distribution of exosomes is conceptually superior to administration of live cells. Other means of isolating exosomes are known in the art, for example, we have previously published techniques for isolation of exosomes from conditioned media and patient plasma [57, 58].

### EXAMPLES

### Example 1

### Stimulation of BM Mononuclear Cell Proliferation by ERC Exosomes

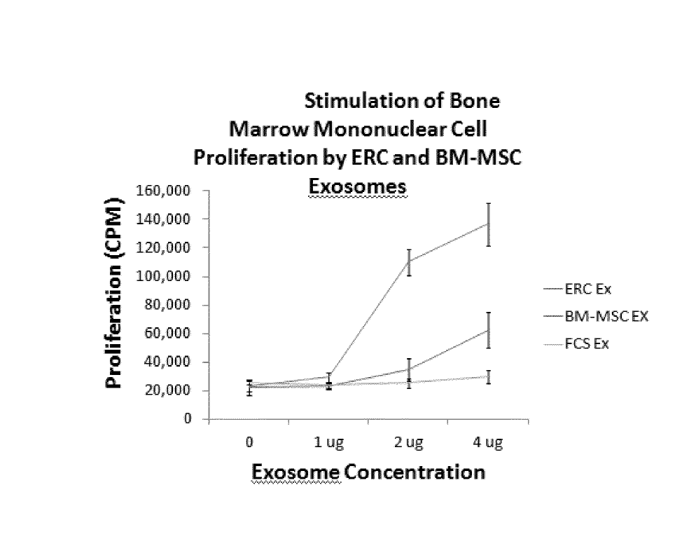

Exosomes were prepared from the supernatant of day 4 ERC or BM-MSC (Cambrex) cultures by differential centrifugation. Conditioned media was subjected to three successive centrifugations at 300 g (5 min), 1,200g (20min), and 10,000g (30 min) to eliminate cells and debris, followed by centrifugation for 1 h at 100,000 g. To remove excess serum proteins, the exosome pellet was washed with a large volume of PBS, centrifuged at 100,000g for 1 h, and resuspended in 120 μl of PBS for further studies. The exosomes were quantified by a micro Bradford protein assay (Bio-Rad). Each batch was standardized by protein content. As a control, we used exosomes isolated from fetal calf serum. To evaluate stem cell stimulatory properties, mouse bone marrow cells were extracted from femurs and tibia of 6-8 week old female C57BL/6 mice (Jackson Laboratories, Bar Harbour, Me.). The bone marrow was triturated using an 18 gauge needle and passed through a 70 μm nylon mesh cell strainer (Becton Dickinson, Franklin Lakes, N.J.) to make a single cell suspension. Bone marrow mononuclear cells were obtained by by gradient centrifugation over Ficoll-Paque (Amersham Pharmacia Biotech, Uppsala, Sweden). Specifically, cells from femurs and tibia of each mouse were pooled and mixed with complete DMEM media in a total volume of 5 ml. 2 ml of Ficoll was layered underneath. Cells were centrifuged for 40 minutes at 600g. The buffy coat was collected and washed 3 times in PBS with 3% fetal calf serum. Bone marrow mononuclear cells were plated at a concentration of 100,000 cells per well in a volume of 100 ml of complete DMEM media. On day 2, 1 uCi of [3H]thymidine was added to each well 16 h before harvest. Radioactive labeling of proliferating T cells was measured on a microplate beta counter (Wallac). Data in FIG. 1 demonstrate that human ERC exosomes possess a higher a stimulatory ability compared to BM-derived exosomes, which in turn was higher than fetal calf serum derived exosomes

### Example 2

### Superior Increased Survival and Hematopoietic Reconstitution Subsequent to Lethal Radiation Exposure by Administration of ERC as Compared to BM-MSC and Adipose MSC

Clinical-grade ERC, are generated according to FDA-approved manufacturing protocols for consistency of phenotype (>90% CD90 and CD105; <5% CD14 and CD34) (passage 6). BM-MSC and adipose derived MSC are purchased from Cambrex and expanded to passage 6. Groups of 10 C57/B6 female mice are subjected to total body irradiation (9.5 Gy) administered using a Cs-137 radiation source as described

. Drinking water is supplemented with neomycin (150 mg/L) and bacitracin (5 mg/L) to minimize infections. Groups receive the following interventions: a) Intravenous infusion of diluent (control); b) 500,000 ERC; c) 1 million ERC; and d) 2 million cells. Cells are administered via tail vein in a volume of 200 uL phosphate buffered saline. Treatment with cells or control is performed at 24 hours post radiation exposure. Additionally, another cohort of animals is treated with the same escalating dose of BM-MSC and adipose derived MSC. Survival and complete blood counts is evaluated over a period of 7 weeks. Blood samples (20 uL) are taken retroorbitally in a vial containing 1 uL of 0.5 M EDTA and cell counts analyzed using a Coulter Onyx. Blood analysis is performed every 3 days. The experiments are performed in triplicate. 100% mortality is observed in the control group, 60% mortality in the BM-MSC group, and approximately the same in the adipose MSC group. 90% survival in the ERC group. Accelerated hematopoietic recovery associated with survival.

### Example 3

### Augmentation of Post Radiation Injury Hematopoietic Recovery by ERC Derived Exosomes

Conditioned media from day 4 of ERC, BM-MSC and adipose MSC, as well as 10% fetal calf serum DMEM media is used as source of exosomes. Isolation of exosomes is performed using the ultracentrifugation method as described in Example 1. Exosome purity is assessed by flow cytometry for the markers CD81, CD63 and CD9 using the previously published method flow cytometry method [59]. Briefly, 10 μl of 4-μm-diameter aldehyde/sulfate latex beads (Interfacial Dynamics, Portland, Oreg., USA) are incubated with purified anti-CD63 mAb (Life Technologies) at room temperature in a small volume (50 μl). After 15 min, the volume is made up to 400 μl with PBS and incubated overnight at 4° C. under gentle agitation; the reaction is stopped by incubation for 30 min in PBS supplemented with 100 mM glycine. For FACS analysis, exosome preparations of 0.05 ug protein or PBS/1%BSA (as negative control) are incubated in 60 for 15 min at 4° C. with anti-CD63-latex beads. The volume is then made up to 400 with PBS and incubated for 2 h at 4° C. Exosome-coated beads are then washed twice in FACS washing buffer (1% BSA and 0.1% NaN3 in PBS) and re-suspended in 400 FACS washing buffer, stained with fluorescent antibodies (CD81, CD63 and CD9) and analyzed on a FACSCalibur flow cytometer (BD Biosciences) and CellQuest software. We found that exosomes purified from conditioned media of ERC, BM-MSC and adipose MSC contained >90% CD81, CD63 and CD9 positive microvesicles which according to our definition are exosomes. Purified exosomes are administered at 24 hours after irradiation (performed as in Example 2) in a volume of 200 uL PBS intravenously. Groups of 10 mice are treated with: a) PBS control; b) 20 ug; c) 40 ug; and d) 80 ug of exosome protein. Exosomes from ERC, BM-MSC and adipose MSC are administered, as well as a comparator group of (5 ng/mouse subcutaneous administration). Quantification of survival and hematological parameters is performed as described in Example 2. 100% mortality is observed in the control group. A dose dependent increase in survival in the ERC, BM-MSC and adipose MSC exosome treated groups, with the lowest mortality (10% dead) in the ERC group. 30-40% is observed in the G-CSF treated group.

### REFERENCES

1. Manthous, C. A. and W. L. Jackson, Jr., The 9-11 Commission's invitation to imagine: a pathophysiology-based approach to critical care of nuclear explosion victims. Critical care medicine, 2007. 35 (3): p. 716-23.

2. Ross, J. R., et al., Radiation injury treatment network (RITN): healthcare professionals preparing for a mass casualty radiological or nuclear incident. International journal of radiation biology, 2011. 87 (8): p. 748-53.

3. Heslet, L., C. Bay, and S. Nepper-Christensen, Acute radiation syndrome (ARS)—treatment of the reduced host defense. International journal of general medicine, 2012. 5: p. 105-15.

4. Herodin, F., et al., Which place for stem cell therapy in the treatment of acute radiation syndrome? Folia histochemica et cytobiologica/Polish Academy of Sciences, Polish Histochemical and Cytochemical Society, 2005. 43 (4): p. 223-7.

5. Kouvaris, J. R., V. E. Kouloulias, and L. J. Vlahos, Amifostine: the first selective-target and broad-spectrum radioprotector. The oncologist, 2007. 12 (6): p. 738-47.

6. Mettler, F. A., Jr., et al., Can radiation risks to patients be reduced without reducing radiation exposure? The status of chemical radioprotectants. AJR. American journal of roentgenology, 2011. 196 (3): p. 616-8.

7. Galotto, M., et al., Stromal damage as consequence of high-dose chemo/radiotherapy in bone marrow transplant recipients. Experimental hematology, 1999. 27 (9): p. 1460-6.

8. Banfi, A., et al., Bone marrow stromal damage after chemo/radiotherapy: occurrence, consequences and possibilities of treatment. Leukemia & lymphoma, 2001. 42 (5): p. 863-70.

9. Almeida-Porada, G., et al., Cotransplantation of human stromal cell progenitors into preimmune fetal sheep results in early appearance of human donor cells in circulation and boosts cell levels in bone marrow at later time points after transplantation. Blood, 2000. 95 (11): p. 3620-7.

10. Noort, W. A., et al., Mesenchymal stem cells promote engraftment of human umbilical cord blood-derived CD34(+) cells in NOD/SCID mice. Experimental hematology, 2002. 30 (8): p. 870-8.

11. Francois, S., et al., Local irradiation not only induces homing of human mesenchymal stem cells at exposed sites but promotes their widespread engraftment to multiple organs: a study of their quantitative distribution after irradiation damage. Stem cells, 2006. 24 (4): p. 1020-9.

12. Zhuo, W., et al., Mesenchymal stem cells ameliorate ischemia-reperfusion-induced renal dysfunction by improving the antioxidant/oxidant balance in the ischemic kidney. Urologia internationalis, 2011. 86 (2): p. 191-6.

13. Nightingale, H., et al., Changes in Expression of the Antioxidant Enzyme SOD3 Occur Upon Differentiation of Human Bone Marrow-Derived Mesenchymal Stem Cells In Vitro. Stem cells and development, 2012.

14. Wei, L., et al., Transplantation of hypoxia preconditioned bone marrow mesenchymal stem cells enhances angiogenesis and neurogenesis after cerebral ischemia in rats. Neurobiology of disease, 2012.

15. Crisostomo, P. R., et al., Human mesenchymal stem cells stimulated by TNF-alpha, LPS, or hypoxia produce growth factors by an NF kappa B-but not JNK-dependent mechanism. American journal of physiology. Cell physiology, 2008. 294 (3): p. C675-82.

16. Wang, M., et al., Human progenitor cells from bone marrow or adipose tissue produce VEGF, HGF, and IGF-I in response to TNF by a p38 MAPK-dependent mechanism. American journal of physiology. Regulatory, integrative and comparative physiology, 2006. 291 (4): p. R880-4.

17. Mouiseddine, M., et al., Human mesenchymal stem cells home specifically to radiation-injured tissues in a non-obese diabetes/severe combined immunodeficiency mouse model. The British journal of radiology, 2007. 80 Spec No 1: p. S49-55.

18. Lazarus, H. M., et al., Ex vivo expansion and subsequent infusion of human bone marrow-derived stromal progenitor cells (mesenchymal progenitor cells): implications for therapeutic use. Bone marrow transplantation, 1995. 16 (4): p. 557-64.

19. Koc, O. N., et al., Rapid hematopoietic recovery after coinfusion of autologous-blood stem cells and culture-expanded marrow mesenchymal stem cells in advanced breast cancer patients receiving high-dose chemotherapy. Journal of clinical oncology: official journal of the American Society of Clinical Oncology, 2000. 18 (2): p. 307-16.

20. Lazarus, H. M., et al., Cotransplantation of HLA-identical sibling culture-expanded mesenchymal stem cells and hematopoietic stem cells in hematologic malignancy patients. Biology of blood and marrow transplantation: journal of the American Society for Blood and Marrow Transplantation, 2005. 11 (5): p. 389-98.

21. Ball, L. M., et al., Cotransplantation of ex vivo expanded mesenchymal stem cells accelerates lymphocyte recovery and may reduce the risk of graft failure in haploidentical hematopoietic stem-cell transplantation. Blood, 2007. 110 (7): p. 2764-7.

22. Baron, F., et al., Cotransplantation of mesenchymal stem cells might prevent death from graft-versus-host disease (GVHD) without abrogating graft-versus-tumor effects after HLA-mismatched allogeneic transplantation following nonmyeloablative conditioning. Biology of blood and marrow transplantation: journal of the American Society for Blood and Marrow Transplantation, 2010. 16 (6): p. 838-47.

23. Tolar, J., et al., Concise review: hitting the right spot with mesenchymal stromal cells. Stem cells, 2010. 28 (8): p. 1446-55.

24. Yang, X., et al., Marrow Stromal Cell Infusion Rescues Hematopoiesis in Lethally Irradiated Mice despite Rapid Clearance after Infusion. Advances in hematology, 2012. 2012: p. 142530.

25. Lange, C., et al., Radiation rescue: mesenchymal stromal cells protect from lethal irradiation. PloS one, 2011. 6 (1): p. e14486.

26. Hu, K. X., et al., The radiation protection and therapy effects of mesenchymal stem cells in mice with acute radiation injury. The British journal of radiology, 2010. 83 (985): p. 52-8.

27. Konchalovsky, M. V., A. E. Baranov, and A. V. Kolganov, Multiple organ involvement and failure: selected Russian radiation accident cases re-visited. BJR supplement/BIR, 2005. 27: p. 26-9.

28. Gale, R. P., Immediate medical consequences of nuclear accidents. Lessons from Chernobyl. JAMA: the journal of the American Medical Association, 1987. 258 (5): p. 625-8.

29. Saha, S., et al., Bone marrow stromal cell transplantation mitigates radiation-induced gastrointestinal syndrome in mice. PloS one, 2011. 6 (9): p. e24072.

30. Honmou, O., et al., Mesenchymal stem cells: therapeutic outlook for stroke. Trends in molecular medicine, 2012.

31. Ali Khalili, M., et al., Therapeutic Benefit of Intravenous Transplantation of Mesenchymal Stem Cells After Experimental Subarachnoid Hemorrhage in Rats. Journal of stroke and cerebrovascular diseases: the official journal of National Stroke Association, 2011.

32. Bao, X., et al., Transplantation of Flk-1+ human bone marrow-derived mesenchymal stem cells promotes angiogenesis and neurogenesis after cerebral ischemia in rats. The European journal of neuroscience, 2011. 34 (1): p. 87-98.

33. Huang, T. T., Redox balance-and radiation-mediated alteration in hippocampal neurogenesis. Free radical research, 2012.

34. Tzouvelekis, A., A. Antoniadis, and D. Bouros, Stem cell therapy in pulmonary fibrosis. Current opinion in pulmonary medicine, 2011. 17 (5): p. 368-73.

35. Nemeth, K., et al., Bone marrow stromal cells attenuate sepsis via prostaglandin E(2)-dependent reprogramming of host macrophages to increase their interleukin-10 production. Nature medicine, 2009. 15 (1): p. 42-9.

36. Meng, X., et al., Endometrial regenerative cells: a novel stem cell population. Journal of translational medicine, 2007. 5: p. 57.

37. Patel, A. N., et al., Multipotent menstrual blood stromal stem cells: isolation, characterization, and differentiation. Cell transplantation, 2008. 17 (3): p. 303-11.

38. Hida, N., et al., Novel cardiac precursor-like cells from human menstrual blood-derived mesenchymal cells. Stem cells, 2008. 26 (7): p. 1695-704.

39. Zhong, Z., et al., Feasibility investigation of allogeneic endometrial regenerative cells. Journal of translational medicine, 2009. 7: p. 15.

40. Ichim, T. E., et al., Mesenchymal stem cells as anti-inflammatories: implications for treatment of Duchenne muscular dystrophy. Cellular immunology, 2010. 260 (2): p. 75-82.

41. Ichim, T. E., et al., Combination stem cell therapy for heart failure. International archives of medicine, 2010. 3 (1): p. 5.

42. Thery, C., M. Ostrowski, and E. Segura, Membrane vesicles as conveyors of immune responses. Nature reviews. Immunology, 2009. 9 (8): p. 581-93.

43. Alonso, R., et al., Diacylglycerol kinase alpha regulates the formation and polarisation of mature multivesicular bodies involved in the secretion of Fas ligand-containing exosomes in T lymphocytes. Cell death and differentiation, 2011. 18 (7): p. 1161-73.

44. Zhang, H., et al., CD4(+) T cell-released exosomes inhibit CD8(+) cytotoxic T-lymphocyte responses and antitumor immunity. Cellular & molecular immunology, 2011. 8 (1): p. 23-30.

45. Mathews, J. A., et al., CD23 Sheddase A disintegrin and metalloproteinase 10 (ADAM10) is also required for CD23 sorting into B cell-derived exosomes. The Journal of biological chemistry, 2010. 285 (48): p. 37531-41.

46. Buschow, S. I., et al., MHC class 11-associated proteins in B-cell exosomes and potential functional implications for exosome biogenesis. Immunology and cell biology, 2010. 88 (8): p. 851-6.

47. Hwang, I. and D. Ki, Receptor-mediated T cell absorption of antigen presenting cell-derived molecules. Frontiers in bioscience: a journal and virtual library, 2011. 16: p. 411-21.

48. Viaud, S., et al., Updated technology to produce highly immunogenic dendritic cell-derived exosomes of clinical grade: a critical role of interferon-gamma. Journal of immunotherapy, 2011. 34 (1): p. 65-75.

49. Clayton, A., et al., Cancer exosomes express CD39 and CD73, which suppress T cells through adenosine production. Journal of immunology, 2011. 187 (2): p. 676-83.

50. Battke, C., et al., Tumour exosomes inhibit binding of tumour-reactive antibodies to tumour cells and reduce ADCC. Cancer immunology, immunotherapy: CII, 2011. 60 (5): p. 639-48.

51. Lachenal, G., et al., Release of exosomes from differentiated neurons and its regulation by synaptic glutamatergic activity. Molecular and cellular neurosciences, 2011. 46 (2): p. 409-18.

52. Faure, J., et al., Exosomes are released by cultured cortical neurones. Molecular and cellular neurosciences, 2006. 31 (4): p. 642-8.

53. Fitzner, D., et al., Selective transfer of exosomes from oligodendrocytes to microglia by macropinocytosis. Journal of cell science, 2011. 124 (Pt 3): p. 447-58.

54. Mincheva-Nilsson, L. and V. Baranov, The role of placental exosomes in reproduction. American journal of reproductive immunology, 2010. 63 (6): p. 520-33.

55. Sahoo, S., et al., Exosomes from human CD34(+) stem cells mediate their proangiogenic paracrine activity. Circulation research, 2011. 109 (7): p. 724-8.

56. Lai, R. C., T. S. Chen, and S. K. Lim, Mesenchymal stem cell exosome: a novel stem cell-based therapy for cardiovascular disease. Regenerative medicine, 2011. 6 (4): p. 481-92.

57. Abusamra, A. J., et al., Tumor exosomes expressing Fas ligand mediate CD8+ T-cell apoptosis. Blood cells, molecules & diseases, 2005. 35 (2): p. 169-73.

58. Ichim, T. E., et al., Exosomes as a tumor immune escape mechanism: possible therapeutic implications. Journal of translational medicine, 2008. 6: p. 37.

59. Caby, M. P., et al., Exosomal-like vesicles are present in human blood plasma. International immunology, 2005. 17 (7): p. 879-87.

What is claimed is:

1. A method for treatment acute radiation syndrome comprising administration of an endometrial derived cell population substantially expressing the markers CD90 and CD105 and substantially lacking the markers CD14, and CD34.

2. The method of claim 1, wherein said acute radiation syndrome is the result of exposure to a radioactive source of 0.5 Gy total body irradiation to 20 Gy total body irradiation.

3. The method of claim 1, wherein said acute radiation syndrome is the result of exposure to a radioactive source of 4 to 12 Gy total body irradiation.

4. The method of claim 1, wherein said endometrially derived cells are administered at a concentration of approximately 5 million to 300 million intravenously.

5. The method of claim 1, wherein said endometrially derived cells are administered at a concentration of approximately 20-200 million intravenously.

6. The method of claim 1, wherein said endometrially derived cells are administered at a concentration of approximately 100 million intravenously.

7. The method of claim 1, wherein said endometrially derived cells are administered at a concentration of approximately 100 million intravenously.

8. A method of treating radiation exposure by administration of mesenchymal stem cell derived exosomes at a sufficient concentration.
